(19) United States
(12) Patent Application Publication
(10) Pub. No.: US 2019/0277862 A1
(43) Pub. Date: Sep. 12, 2019

(54) EVALUATING METHOD FOR MILD COGNITIVE IMPAIRMENT OR ALZHEIMER'S DISEASE

(71) Applicant: AJINOMOTO CO., INC., Tokyo (JP)

(72) Inventors: Takeshi IKEUCHI, Niigata (JP); Nobuhiro KAWAI, Kanagawa (JP); Yuki YANO, Kanagawa (JP); Naoko ARASHIDA, Kanagawa (JP); Rumi NISHIMOTO, Kanagawa (JP); Kazutaka SHIMBO, Kanagawa (JP)

(73) Assignee: AJINOMOTO CO., INC., Tokyo (JP)

(21) Appl. No.: 16/239,817

(22) Filed: Jan. 4, 2019

Related U.S. Application Data
(63) Continuation of application No. PCT/JP2017/025051, filed on Jul. 7, 2017.

(30) Foreign Application Priority Data
Jul. 8, 2016 (JP) 2016-136340

(51) Int. Cl. G01N 33/68

(52) U.S. Cl. CPC G01N 33/6896; G01N 2800/2821; G01N 2800/2814

(57) ABSTRACT

An evaluating method includes an evaluating step of evaluating a state of mild cognitive impairment or Alzheimer's disease for a subject to be evaluated using a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, GABA, Hypotaurine, S-Adenosyl homocysteine, Sarcosine, Serotonin, Spermidine, hCit, 3-hKyn, aAiBA, N8-Acetylspermidine, Phosphoserine, bABA, ADMA, and Thioproline in blood of the subject.

### CROSS-REFERENCE TO RELATED APPLICATION

This application is based upon and claims the benefit of priority from PCT Application PCT/JP2017/025051, filed Jul. 7, 2017, which claims priority from Japanese Patent Application No. 2016-136340, filed Jul. 8, 2016, the entire contents of which are incorporated herein by reference.

### BACKGROUND OF THE INVENTION

### 1. Field of the Invention

The present invention relates to an evaluating method, an evaluating apparatus, an evaluating program product, an evaluating system, and a terminal apparatus for mild cognitive impairment (hereinafter, may be referred to as MCI) in the prodromal stage of dementia, or Alzheimer's disease (hereinafter, may be referred to as AD).

### 2. Description of the Related Art

Dementia is a condition where the normally developed intellectual function of a patient has been entirely and continuously decreased by acquired brain lesions and the daily living of the patient is affected, and is a disease defined as “a syndrome generally caused by chronic or progressive brain disease, comprising a plurality of disabled higher cerebral functions of memory, thought, orientation, comprehension, calculation, learning, language, and decision” (“International Classification of Diseases, 10th revision, World Health Organization”). It is said that 60% of primary causes of dementia is Alzheimer's disease that is most common.

The typical neuropathological features of AD are neuritic plaques and neurofibrillary tangles. It has been found that the neuritic plaques are caused by deposition of the protein called amyloid β (Aβ) and the neurofibrillary tangles are caused by excessively phosphorylated tau protein. A recent large-scale observation study revealed that these pathological features appear before AD develops (“Jack, et al., Lancet Neurol (2010) 9(1): 119-28; Hypothetical model of dynamic biomarkers of the Alzheimer's pathological cascade.”). In the recent years, some diagnostic imaging techniques, such as the positron emission tomography (PET), the single photon emission computed tomography (SPECT), and the magnetic resonance imaging (MRI), have been offered as methods for quantifying the accumulation of Aβ, and phosphorylated tau protein in brain tissue and quantifying the atrophy of brain tissue. However, independent use of any one of the diagnostic imaging techniques mentioned above is not recommended as a definitive diagnosis method. At present, AD is diagnosed based on total evaluation including a neuropsychological test and clinical symptom observation. Besides, a technique for diagnosing AD using the concentrations of Aβ, and phosphorylated tau protein in cerebrospinal fluid (CSF) as indices has been offered (“Knopman, et al., Neurology. (2001) 8; 56(9): 1143-53; Practice parameter: diagnosis of dementia (an evidence-based review). Report of the Quality Standards Subcommittee of the American Academy of Neurology.”).

These techniques, however, are expensive and highly invasive, and therefore, they are not suitable for mass screening test executable in, for example, medical checkup. Currently, the neuropsychological test widely spread as a screening test for dementia takes a long time for examination and involves a skilled examiner, and thus a limited number of medical institutions can execute the neuropsychological test. Thus, it has been desired that a simple blood test be established at a lower cost.

The acetylcholinesterase inhibitor and the N-methyl-D-aspartic acid (NMDA) receptor inhibitor are used as a therapeutic agent against AD, but those medicines can merely exert the effect of delaying progression of the disease for a certain period, and a disease modifying therapy necessary for the radical cure has not been established yet. In addition, antibody drugs have been developed based on neuropathological findings in the accumulation of Aβ, and phosphorylated tau protein, but candidate medicines exerting remarkable effects have not been found yet.

On these backgrounds, the necessities of early diagnostic before developing AD and prevention of development of AD through the early intervention have increased in recent years. In the present description, mild cognitive impairment means a condition considered as a previous stage or a borderline case of various types of dementia that shows severer problems in cognitive function than those of a person of the same age or a normally aging person, but does not affect daily living, being not considered as dementia. At present, the major two diagnostic criteria have been offered and widely accepted (“Petersen, et al., Arch Neurol (1999) 56(6):760; Mild cognitive impairment: clinical characterization and outcome.” and “Winblad, et al., J. Intern. Med. (2004) 256(3): 240-6; Mild cognitive impairment—beyond controversies, towards a consensus: report of the International Working Group on Mild Cognitive Impairment.”). It has been revealed that a number of patients diagnosed as MCI develop AD in several years at high probabilities. Therefore, it has been desired to provide simple screening methods that can diagnose MCI to increase the chance for more subjects to have a medical checkup in earlier stages at specialized institutions, to increase the chance for more subjects to get a treatment opportunity in earlier stages, and to contribute to avoiding increase in the number of patients of dementia.

Unfortunately, when the neuropsychological test called “mini mental state examination (MMSE)”, the revised Hasegawa dementia scale “HDS-R”, and the Alzheimer's disease assessment scale-cognitive (ADAS-cog) that are spread as screening test methods for dementia such as AD are applied to screening for MCI, it is known that the accuracy of detecting MCI is decreased relative to the accuracy of detecting dementia. Thus, it has been desired to provide a new and simple test technique as an MCI screening test method to aid a conventional neuropsychological test.

Incidentally, some methods of measuring the blood concentration of amino acids and amino acid related metabolites to determine incidence risk based on the features in a particular disease have been known in the diagnosis of diseases such as cancer, metabolic syndrome, and liver disease (JP-A-2014-025946, JP-A-2016-029398, and JP-A-2013-040923). A technique for diagnosing AD based on the blood concentrations of particular amino acids as indices has been developed (JP-A-2011-242217). As a technique for determining MCI based on a blood test, a technique for measuring the blood concentration of peptide fragments for use as indices has been developed (JP-A-2016-028244).

However, there is a problem in that techniques for accurately determining AD and MCI, based on the blood concentration of amino acids and amino acid related metabolites as indices, have not been developed or put into practice yet.

### SUMMARY OF THE INVENTION

It is an object of the present invention to at least partially solve the problems in the conventional technology.

The present invention has been made in view of the above descriptions, and an object of the present invention is to provide an evaluating method, an evaluating apparatus, an evaluating program product, an evaluating system, and a terminal apparatus, which can provide reliable information that may be helpful in knowing a state of MCI or AD.

To solve the problem and achieve the object described above, an evaluating method according to one aspect of the present invention includes an evaluating step of evaluating a state of mild cognitive impairment or Alzheimer's disease for a subject to be evaluated using a concentration value of at least one of 23 kinds of amino acids (α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine) and 14 kinds of amino acid related metabolites (11 kinds of amino Acid related metabolites (GABA [4-Aminobutyric Acid], Hypotaurine, S-Adenosyl homocysteine, Sarcosine, Serotonin, Spermidine, hcit [L-Homocitrulline], 3-hkyn [3-Hydroxy-L-Kynurenine], aaiba [2-Aminoisobutyric acid], N8-Acetylspermidine, Phosphoserine) with three kinds of amino Acid related metabolites (baba [3-Aminobutanoic acid], ADMA [Asymmetric dimethylarginine], Thioproline)) in blood of the subject.

In the present description, various amino acids are mainly written in abbreviations, the formal names of these are as follows.

| (Abbreviation) | (Formal name) |
|---|---|
| α-ABA | α-Aminobutyric acid |
| Ala | Alanine |
| Arg | Arginine |
| Asn | Asparagine |
| Cit | Citrulline |
| Gln | Glutamine |
| Glu | Glutamic acid |
| Gly | Glycine |
| His | Histidine |
| Ile | Isoleucine |
| Leu | Leucine |
| Lys | Lysine |
| Met | Methionine |
| Orn | Ornithine |
| Phe | Phenylalanine |
| Pro | Proline |
| Ser | Serine |
| Thr | Threonine |
| Trp | Tryptophan |
| Tyr | Tyrosine |
| Val | Valine |

An evaluating apparatus according to one aspect of the present invention is an evaluating apparatus including a control unit. The control unit includes an evaluating unit that evaluates a state of mild cognitive impairment or Alzheimer's disease for a subject to be evaluated using a concentration value of at least one of the 23 kinds of amino acids and the 14 kinds of amino acid related metabolites in blood of the subject.

An evaluating method according to one aspect of the present invention is an evaluating method executed by an information processing apparatus including a control unit. The evaluating method includes an evaluating step of evaluating a state of mild cognitive impairment or Alzheimer's disease for a subject to be evaluated using a concentration value of at least one of the 23 kinds of amino acids and the 14 kinds of amino acid related metabolites in blood of the subject. The evaluating step is executed by the control unit.

An evaluating program product according to one aspect of the present invention is an evaluating program product having a non-transitory tangible computer readable medium including a programmed instruction for making an information processing apparatus including a control unit execute an evaluating method. The evaluating method includes an evaluating step of evaluating a state of mild cognitive impairment or Alzheimer's disease for a subject to be evaluated using a concentration value of at least one of the 23 kinds of amino acids and the 14 kinds of amino acid related metabolites in blood of the subject. The evaluating step is executed by the control unit.

A recording medium according to one aspect of the present invention is a non-transitory tangible computer-readable recording medium including a programmed instruction for making an information processing apparatus execute the evaluating method.

An evaluating system according to one aspect of the present invention is an evaluating system including an evaluating apparatus including a control unit and a terminal apparatus including a control unit that are connected to each other communicatively via a network. The control unit of the terminal apparatus includes (i) a concentration data-sending unit that transmits concentration data on a concentration value of at least one of the 23 kinds of amino acids and the 14 kinds of amino acid related metabolites in blood of a subject to be evaluated, to the evaluating apparatus and (ii) a result-receiving unit that receives an evaluation result on a state of mild cognitive impairment or Alzheimer's disease for the subject, transmitted from the evaluating apparatus. The control unit of the evaluating apparatus includes (i) a concentration data-receiving unit that receives the concentration data transmitted from the terminal apparatus, (ii) an evaluating unit that evaluates the state of mild cognitive impairment or Alzheimer's disease for the subject using the concentration value of the at least one of the amino acids and the amino acid related metabolites included in the concentration data received by the concentration data-receiving unit, and (iii) a result-sending unit that transmits the evaluation result obtained by the evaluating unit to the terminal apparatus.

A terminal apparatus according to one aspect of the present invention is a terminal apparatus including a control unit. The control unit includes a result-obtaining unit that obtains an evaluation result on a state of mild cognitive impairment or Alzheimer's disease for a subject to be evaluated. The evaluation result is the result of evaluating the state of mild cognitive impairment or Alzheimer's disease for the subject using a concentration value of at least one of the 23 kinds of amino acids and the 14 kinds of amino acid related metabolites in blood of the subject.

The terminal apparatus according to another aspect of the present invention is the terminal apparatus, wherein the apparatus is communicatively connected via a network to an evaluating apparatus that evaluates the state of mild cognitive impairment or Alzheimer's disease for the subject. The control unit further includes a concentration data-sending unit that transmits concentration data on the concentration value of the at least one of the amino acids and the amino acid related metabolites to the evaluating apparatus. The result-obtaining unit receives the evaluation result transmitted from the evaluating apparatus.

An evaluating apparatus according to one aspect of the present invention is an evaluating apparatus including a control unit, being connected to a terminal apparatus communicatively via a network. The control unit includes (i) a concentration data-receiving unit that receives concentration data on a concentration value of at least one of the 23 kinds of amino acids and the 14 kinds of amino acid related metabolites in blood of a subject to be evaluated, transmitted from the terminal apparatus, (ii) an evaluating unit that evaluates a state of mild cognitive impairment or Alzheimer's disease for the subject using the concentration value of the at least one of the amino acids and the amino acid related metabolites included in the concentration data received by the concentration data-receiving unit, and (iii) a result-sending unit that transmits an evaluation result obtained by the evaluating unit to the terminal apparatus.

According to the present invention, reliable information that may be helpful in knowing the state of mild cognitive impairment or Alzheimer's disease can be provided.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a principle configurational diagram showing a basic principle of a first embodiment;

FIG. 2 is a principle configurational diagram showing a basic principle of a second embodiment;

FIG. 3 is a diagram showing an example of an entire configuration of a present system;

FIG. 4 is a block diagram showing an example of a configuration of an evaluating apparatus 100 in the present system;

FIG. 5 is a chart showing an example of information stored in a concentration data file 106a;

FIG. 6 is a chart showing an example of information stored in an evaluation result file 106b;

FIG. 7 is a block diagram showing a configuration of an evaluating part 102b; and

FIG. 8 is a block diagram showing an example of a configuration of a client apparatus 200 in the present system.

### DETAILED DESCRIPTION OF THE PREFERRED EMBODIMENTS

Hereinafter, an embodiment (first embodiment) of the evaluating method according to the present invention and an embodiment (second embodiment) of the evaluating apparatus, the evaluating method, the evaluating program product, the recording medium, the evaluating system, and the terminal apparatus according to the present invention are described in detail with reference to the drawings. The present invention is not limited to these embodiments.

### First Embodiment

### 1-1. Outline of First Embodiment

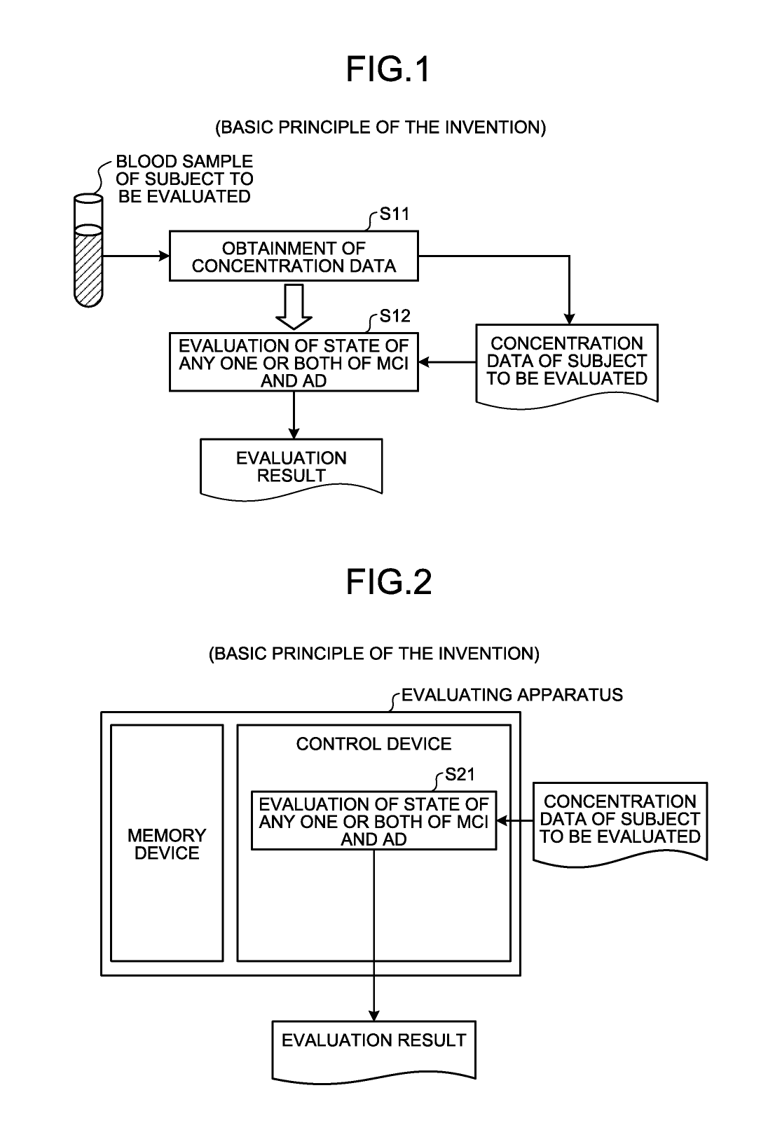

Here, an outline of the first embodiment will be described with reference to FIG. 1. FIG. 1 is a principle configurational diagram showing a basic principle of the first embodiment.

First, concentration data on a concentration value of at least one of the 23 kinds of amino acids and the 14 kinds of amino acid related metabolites (a substance or substances arbitrarily selected from the 23 kinds of amino acids and the 14 kinds of amino acid related metabolites) contained in the blood (including, for example, plasma or serum) extracted from a subject to be evaluated (for example, an individual such as animal or human) is obtained (Step S11).

At step S11, for example, the concentration data measured by a company or other organization that measures concentrations may be obtained. In addition, for example, the following measuring method of (A), (B), or (C) may be used to measure the concentration from the blood extracted from the subject to obtain the concentration data. In the present description, the unit of the concentration may be molar concentration, weight concentration, enzyme activity, or one obtained by addition, subtraction, multiplication, and division of any constant with these concentrations. When the measuring method of (A) is used, the peak area or the peak height of each substance in a chromatogram obtained by a mass spectrometer may be used as a substitute for a concentration.

(A) Plasma is separated from blood by centrifuging the collected blood sample. All plasma samples are frozen and stored at −80° C. until the concentration is measured. At the time of measuring the concentration, acetonitrile is added to deproteinize the plasma samples, pre-column derivatization is then performed using a labeling reagent (3-aminopyridyl-N-hydroxysuccinimidyl carbamate), and the concentration is analyzed by liquid chromatograph mass spectrometer (LC/MS) (see International Publication WO 2003/069328 and International Publication WO 2005/116629).

(B) Plasma is separated from blood by centrifuging the collected blood sample. All plasma samples are frozen and stored at −80° C. until the concentration is measured. At the time of measuring the concentration, sulfosalicylic acid is added to deproteinize the plasma samples, and the concentration is analyzed by an amino acid analyzer based on post-column derivatization using a ninhydrin reagent.

(C) Blood cell separation is performed on the collected blood sample by using a membrane, MEMS (Micro Electro Mechanical Systems) technology, or the principle of centrifugation, whereby plasma or serum is separated from the blood. A plasma or serum sample the concentration of which is not measured immediately after obtaining the plasma or the serum is frozen and stored at −80° C. until the concentration is measured. At the time of measuring the concentration, a molecule that reacts with or binds to a target amino acid or amino acid related metabolite, such as an enzyme or an aptamer, and the like are used to perform quantitative analysis and the like on an increasing or decreasing substance or a spectroscopic value by substrate recognition, whereby the concentration is analyzed.

The state of any one or both of MCI and AD for the subject is evaluated using the concentration value of at least one of the 23 kinds of amino acids and the 14 kinds of amino acid related metabolites included in the concentration data obtained at step S11 (step S12). Before step S12 is executed, data such as defective and outliers may be removed from the concentration data obtained at step S11. In the present description, evaluation of a state is, for example, to examine the present condition.

According to the first embodiment described above, the concentration data of the subject is obtained at step S11, and at step S12, the state of any one or both of MCI and AD for the subject is evaluated using the concentration value of at least one of the 23 kinds of amino acids and the 14 kinds of amino acid related metabolites included in the concentration data of the subject obtained at step S11. Hence, reliable information that may be helpful in knowing the state of any one or both of MCI and AD can be provided. The evaluating method according to the present embodiment is valuable as a simple and inexpensive method of determining and testing any one or both of MCI and AD suitable for mass screening.

It may be decided that the concentration value of at least one of the 23 kinds of amino acids and the 14 kinds of amino acid related metabolites reflects the state of any one or both of MCI and AD for the subject. The concentration value may be converted, for example, by the methods listed below, and it may be decided that the converted value reflects the state of any one or both of MCI and AD for the subject. In other words, the concentration value or the converted value may be treated per se as an evaluation result on the state of any one or both of MCI and AD for the subject.

The concentration value may be converted such that a possible range of the concentration value falls within a predetermined range (for example, the range from 0.0 to 1.0, the range from 0.0 to 10.0, the range from 0.0 to 100.0, or the range from −10.0 to 10.0), for example, by addition, subtraction, multiplication, and division of any given value with the concentration value, by conversion of the concentration value by a predetermined conversion method (for example, exponential transformation, logarithm transformation, angular transformation, square root transformation, probit transformation, reciprocal transformation, Box-Cox transformation, or power transformation), or by performing a combination of these computations on the concentration value. For example, a value of an exponential function with the concentration value as an exponent and Napier constant as the base may be further calculated (specifically, a value of p/(1−p) where a natural logarithm ln(p/(1−p)) is equal to the concentration value when the probability p that the state of any one or both of MCI and AD has a predetermined state (for example, a state of having any one or both of MCI and AD with a high probability) is defined), and a value (specifically, a value of the probability p) may be further calculated by dividing the calculated value of the exponential function by the sum of 1 and the value of the exponential function.

The concentration value may be converted such that the converted value is a particular value when a particular condition is met. For example, the concentration value may be converted such that the converted value is 5.0 when the sensitivity is 95% and the converted value is 8.0 when the sensitivity is 80%.

For each amino acid and each amino acid related metabolite, after normally distributing the concentration distribution, the concentration value may be standardized with a mean of 50 and a standard deviation of 10.

These conversions may be performed by gender or age.

The state of any one or both of MCI and AD for the subject may be evaluated using the concentration value converted, for example, by the conversion method described above.

Positional information about a position of a predetermined mark on a predetermined scale visually presented on a display device such as a monitor or a physical medium such as paper may be generated using the concentration value of at least one of the 23 kinds of amino acids and the 14 kinds of amino acid related metabolites or, if the concentration value is converted, the converted value, and it may be decided that the generated positional information reflects the state of any one or both of MCI and AD for the subject. The predetermined scale is for evaluating the state of any one or both of MCI and AD and is, for example, a graduated scale at least marked with graduations corresponding to the upper limit value and the lower limit value in “a possible range of the concentration value or the converted value”, or “part of the range”. The predetermined mark corresponds to the concentration value or the converted value and is, for example, a circle sign or a star sign.

If the concentration value of at least one of the 23 kinds of amino acids and the 14 kinds of amino acid related metabolites is lower than a predetermined value (e.g., mean±1SD, 2SD, 3SD, N quantile, N percentile, or a cutoff value the clinical significance of which is recognized) or is equal to or lower than the predetermined value, or the concentration value is equal to or higher than the predetermined value or is higher than the predetermined value, the state of any one or both of MCI and AD for the subject may be evaluated. In this case, instead of the concentration value itself, a standard score (a value obtained by normally distributing the concentration distribution by gender and then standardizing the concentration value with a mean of 50 and a standard deviation of 10 for each amino acid and each amino acid related metabolite) may be used. For example, if the concentration standard score is lower than the mean−2SD (when the concentration standard score<30) or if the concentration standard score is higher than the mean+2SD (when the concentration standard score>70), the state of any one or both of MCI and AD for the subject may be evaluated.

The degree of the possibility that the subject has any one or both of MCI and AD may be qualitatively evaluated. Specifically, the subject may be classified into any one of a plurality of categories defined at least considering the degree of the possibility of having any one or both of MCI and AD, using the concentration value of at least one of the 23 kinds of amino acids and the 14 kinds of amino acid related metabolites, and one or more preset thresholds. The categories may include (i) a category to which a subject with a high degree of the possibility of having any one or both of MCI and AD (for example, a subject assumed to have any one or both of MCI and AD) belongs, (ii) a category to which a subject with a low degree of the possibility of having any one or both of MCI and AD (for example, a subject assumed not to have MCI or AD) belongs, and (iii) a category to which a subject with an intermediate degree of the possibility of having any one or both of MCI and AD belongs. The categories may include (i) the category to which a subject with a high degree of the possibility of having any one or both of MCI and AD belongs, and (ii) the category to which a subject with a low degree of the possibility of having any one or both of MCI and AD belongs. The concentration value may be converted by the predetermined method, and the subject may be classified into any one of the categories using the converted value.

When the state of any one or both of MCI and AD is evaluated, a value related to other biological information listed below may further be used in addition to the concentration value of at least one of the 23 kinds of amino acids and the 14 kinds of amino acid related metabolites.

1. Concentration values of metabolites in blood other than amino acids (e.g., amino acid metabolites, carbohydrates, and lipids), proteins, peptides, minerals, vitamins, organic acids, hormones, and the like.
2. Blood test values such as albumin, total protein, triglyceride (neutral fat), HbAlc, glycoalbumin, insulin resistance index, total cholesterol, LDL cholesterol, HDL cholesterol, amylase, total bilirubin, creatinine, estimated glomerular filtration rate (eGFR), uric acid, GOT (AST), GPT (ALT), GGTP (γ-GTP), glucose (glucose level), CRP (C-reactive protein), erythrocyte, hemoglobin, hematocrit, MCV, MCH, MCHC, leucocyte, and the number of thrombocytes.
3. Values obtained from image information such as ultrasonic echo, X ray, CT (Computed Tomography), MRI (Magnetic Resonance Imaging), and endoscope image.

4. Values of biological indices such as age, height, weight, BMI, abdominal girth, systolic blood pressure, diastolic blood pressure, gender, smoking information, dietary information, drinking information, exercise information, stress information, sleeping information, family medical history information, and disease history information (for example, diabetes).

5. Values obtained from gene information, for example, number of risk genes for Alzheimer's disease (for example, APOEε4 allele).

### Second Embodiment

### 2-1. Outline of the Second Embodiment

Here, outlines of the second embodiment will be described in detail with reference to FIG. 2. FIG. 2 is a principle configurational diagram showing a basic principle of the second embodiment. In the description of the present second embodiment, description duplicating that of the first embodiment is sometimes omitted.

A control device evaluates the state of any one or both of MCI and AD for the subject using the concentration value of at least one of the 23 kinds of amino acids and the 14 kinds of amino acid related metabolites included in the previously obtained concentration data of the subject (for example, an individual such as animal or human) on the concentration value of at least one of the 23 kinds of amino acids and the 14 kinds of amino acid related metabolites in blood (step S21). Hence, reliable information that may be helpful in knowing the state of any one or both of MCI and AD can be provided.

### 2-2. System Configuration

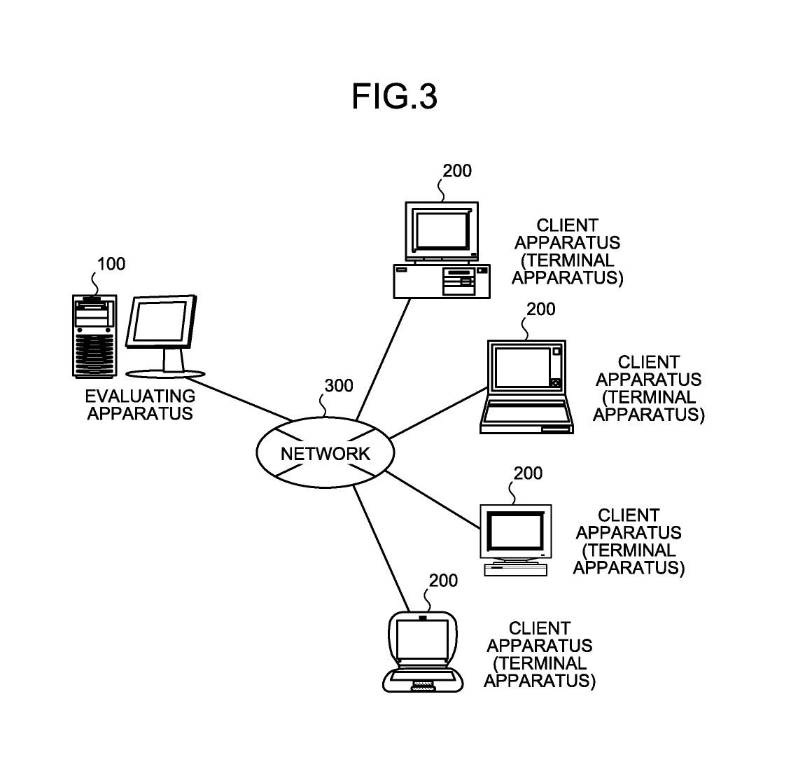

Hereinafter, the configuration of the evaluating system according to the second embodiment (hereinafter referred to sometimes as the present system) will be described with reference to FIGS. 3 to 8. This system is merely one example, and the present invention is not limited thereto.

First, an entire configuration of the present system will be described with reference to FIG. 3. FIG. 3 is a diagram showing an example of the entire configuration of the present system. As shown in FIG. 3, the present system is constituted in which the evaluating apparatus 100 that evaluates the state of any one or both of MCI and AD for the individual as the subject and the client apparatus 200 (corresponding to the terminal apparatus of the present invention) that provides the concentration data of the individual are communicatively connected to each other via a network 300.

The network 300 has a function to connect the evaluating apparatus 100 and the client apparatuses 200 mutually, communicatively to one another, and is for example the Internet, an intranet, or LAN (Local Area Network (including both wired and wireless)). The network 300 may be VAN (Value Added Network), a personal computer communication network, a public telephone network (including both analog and digital), a leased line network (including both analog and digital), CATV (Community Antenna Television) network, a portable switched network or a portable packet-switched network (including IMT2000 (International Mobile Telecommunication 2000) system, GSM (registered trademark) (Global System for Mobile Communications) system, or PDC (Personal Digital Cellular)/PDC-P system), a wireless calling network, a local wireless network such as Bluetooth (registered trademark), PHS network, a satellite communication network (including CS (Communication Satellite), BS (Broadcasting Satellite), ISDB (Integrated Services Digital Broadcasting), and the like), or the like.

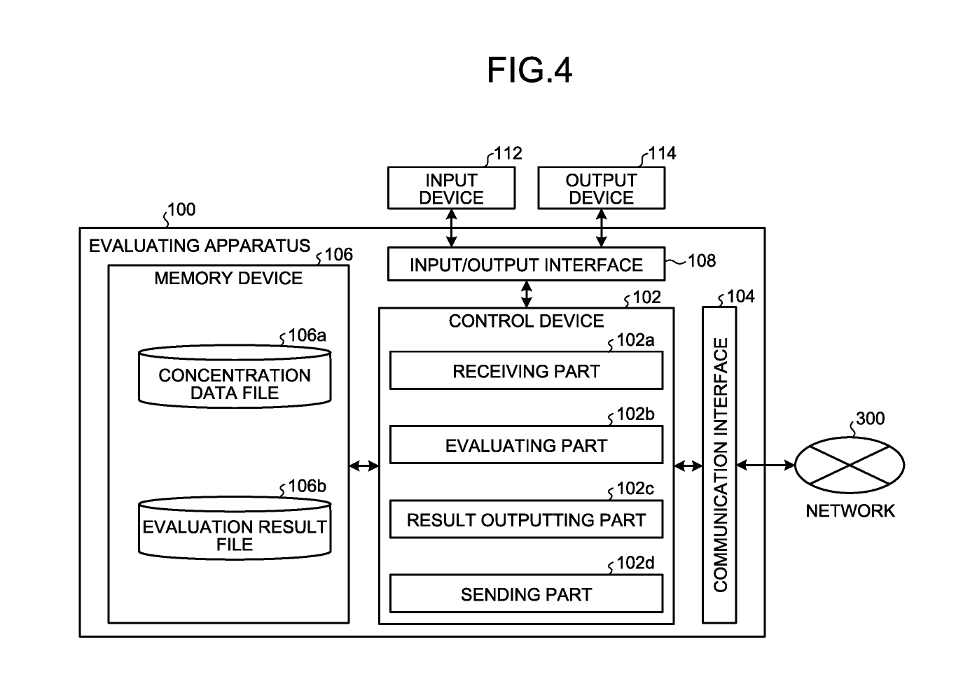

Now, the configuration of the evaluating apparatus 100 in the present system will be described with reference to FIGS. 4 to 7. FIG. 4 is a block diagram showing an example of the configuration of the evaluating apparatus 100 in the present system, showing conceptually only the region relevant to the present invention.

The evaluating apparatus 100 includes (i) a control device 102, such as CPU (Central Processing Unit), that integrally controls the evaluating apparatus, (ii) a communication interface 104 that connects the evaluating apparatus to the network 300 communicatively via communication apparatuses such as a router and wired or wireless communication lines such as a private line, (iii) a memory device 106 that stores various databases, tables, files and others, and (iv) an input/output interface 108 connected to an input device 112 and an output device 114, and these parts are connected to each other communicatively via any communication channel. The evaluating apparatus 100 may be present together with various analyzers (e.g., an amino acid analyzer) in a same housing. For example, the evaluating apparatus 100 may be a compact analyzing device including components (hardware and software) that calculate (measure) the concentration value of at least one of the 23 kinds of amino acids and the 14 kinds of amino acid related metabolites in blood and output (e.g., print or display on a monitor) the calculated concentration value, wherein the compact analyzing device is characterized by further including the evaluating part 102b described later, and using the components to output results obtained by the evaluating part 102b.

The communication interface 104 allows communication between the evaluating apparatus 100 and the network 300 (or a communication apparatus such as a router). Thus, the communication interface 104 has a function to communicate data via a communication line with other terminals.

The input/output interface 108 is connected to the input device 112 and the output device 114. A monitor (including a home television), a speaker, or a printer may be used as the output device 114 (hereinafter, the output device 114 may be described as the monitor 114). A keyboard, a mouse, a microphone, or a monitor functioning as a pointing device together with a mouse may be used as the input device 112.

The memory device 106 is a storage means, and examples thereof include a memory apparatus such as RAM (Random Access Memory) and ROM (Read Only Memory), a fixed disk drive such as a hard disk, a flexible disk, and an optical disk. The memory device 106 stores computer programs giving instructions to the CPU for various processings, together with OS (Operating System). As shown in the figure, the memory device 106 stores the concentration data file 106a and the evaluation result file 106b.

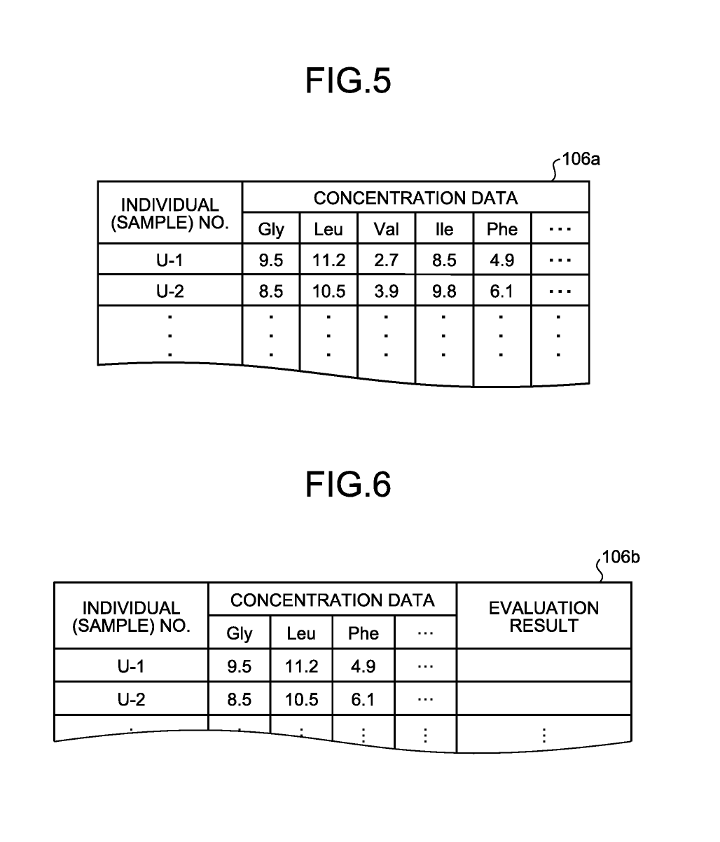

The concentration data file 106a stores the concentration value of at least one of the 23 kinds of amino acids and the 14 kinds of amino acid related metabolites in blood. FIG. 5 is a chart showing an example of information stored in the concentration data file 106a. As shown in FIG. 5, the information stored in the concentration data file 106a includes an individual number for uniquely identifying the individual (sample) as the subject and the concentration data that are correlated to one another. In FIG. 5, the concentration data is assumed to be numerical values, i.e., on a continuous scale, but the concentration data may be expressed on a nominal scale or an ordinal scale. In the case of the nominal or ordinal scale, any number may be allocated to each state for analysis. The concentration data may be combined with the value related to the other biological information.

Returning to FIG. 4, the evaluation result file 106b stores the evaluation results obtained in the evaluating part 102b described below. FIG. 6 is a chart showing an example of information stored in the evaluation result file 106b. The information stored in the evaluation result file 106b includes the individual number for uniquely identifying the individual (sample) as the subject, the previously obtained concentration data of the individual, and the evaluation result on the state of any one or both of MCI and AD (for example, the converted value of the concentration value by a converting part 102b1 described below, the positional information generated by a generating part 102b2 described below, or the classification result obtained by a classifying part 102b3 described below), that are correlated to one another.

Returning to FIG. 4, the control device 102 has an internal memory storing, for example, control programs such as OS (Operating System), programs for various processing procedures, and other needed data, and performs various information processings according to these programs. As shown in the figure, the control device 102 includes mainly a receiving part 102a, the evaluating part 102b, a result outputting part 102c, and a sending part 102d. The control device 102 performs data processings such as removal of data including defective, removal of data including many outliers, and removal of explanatory variables for the defective-including data in the concentration data transmitted from the client apparatus 200.

The receiving part 102a receives, via the network 300, information (specifically, the concentration data, etc.) transmitted from the client apparatus 200.

The evaluating part 102b evaluates the state of any one or both of MCI and AD for the individual using the concentration value of at least one of the 23 kinds of amino acids and the 14 kinds of amino acid related metabolites included in the concentration data of the individual received by the receiving part 102a.

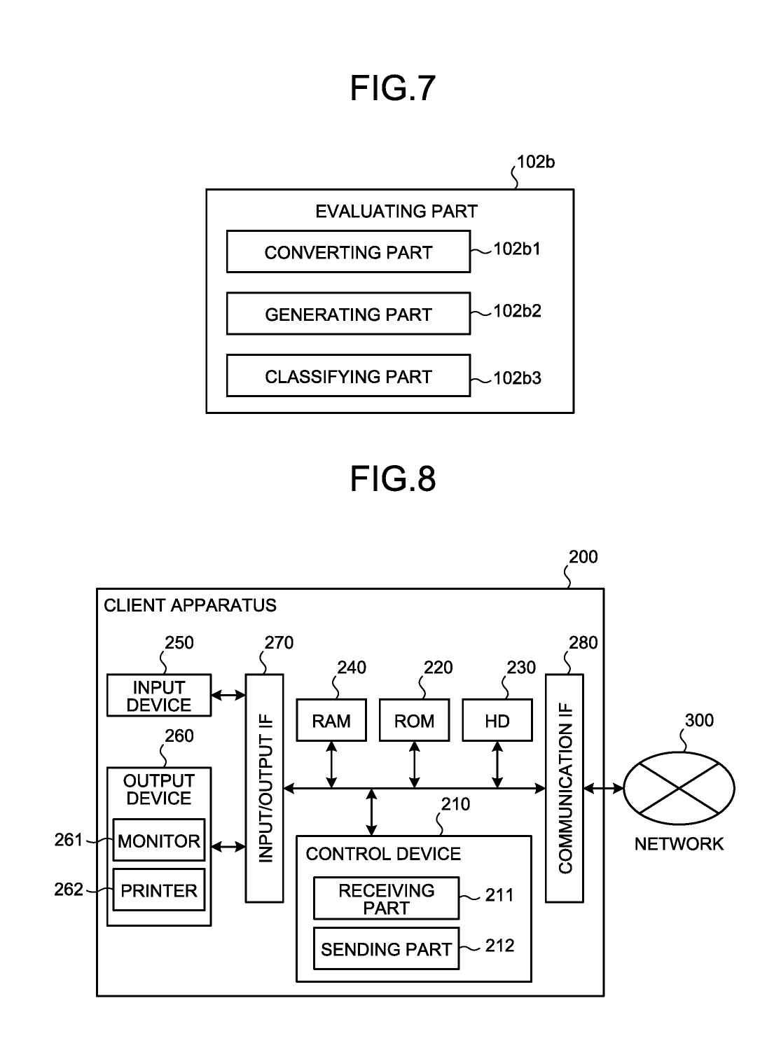

Hereinafter, a configuration of the evaluating part 102b will be described with reference to FIG. 7. FIG. 7 is a block diagram showing the configuration of the evaluating part 102b, and only a part in the configuration related to the present invention is shown conceptually. The evaluating part 102b includes the converting part 102b1, the generating part 102b2, and the classifying part 102b3, additionally.

The converting part 102b1 converts the concentration value of at least one of the 23 kinds of amino acids and the 14 kinds of amino acid related metabolites included in the concentration data, for example, by the conversion method described above. The evaluating part 102b may store the converted value by the converting part 102b1 as the evaluation result in a predetermined region of the evaluation result file 106b.

The generating part 102b2 generates the positional information about the position of the predetermined mark on the predetermined scale visually presented on the display device such as a monitor or the physical medium such as paper, using the concentration value or the converted value of the concentration value by the converting part 102b1. The evaluating part 102b may store the positional information generated by the generating part 102b2 as the evaluation result in a predetermined region of the evaluation result file 106b.

The classifying part 102b3 classifies the individual into any one of the categories defined at least considering the degree of the possibility of having any one or both of MCI and AD, using the concentration value or the converted value of the concentration value by the converting part 102b1.

The result outputting part 102c outputs, into the output device 114, for example, the processing results in each processing part in the control device 102 (including the evaluation results obtained by the evaluating part 102b).

The sending part 102d is a means for performing data transmission to an external apparatus and, for example, transmits the evaluation results obtained by the evaluating part 102b to the client apparatus 200 that is a sender of the concentration data of the individual.

Hereinafter, a configuration of the client apparatus 200 in the present system will be described with reference to FIG. 8. FIG. 8 is a block diagram showing an example of the configuration of the client apparatus 200 in the present system, and only the part in the configuration relevant to the present invention is shown conceptually.

The client apparatus 200 includes a control device 210, ROM 220, HD (Hard Disk) 230, RAM 240, an input device 250, an output device 260, an input/output IF 270, and a communication IF 280 that are connected communicatively to one another through a communication channel.

The control device 210 has a receiving part 211 and a sending part 212. The receiving part 211 receives various kinds of information such as the evaluation results transmitted from the evaluating apparatus 100, via the communication IF 280. The sending part 212 sends various kinds of information such as the concentration data of the individual, via the communication IF 280, to the evaluating apparatus 100. The control device 210 may include an evaluating part 210a (including a converting part 210a1, a generating part 210a2, and a classifying part 210a3) having the same functions as the functions of the evaluating part 102b in the control device 102 of the evaluating apparatus 100.

The input device 250 is, for example, a keyboard, a mouse, or a microphone. The monitor 261 described below also functions as a pointing device together with a mouse. The output device 260 is an output means for outputting information received via the communication IF 280, and includes the monitor 261 (including home television) and a printer 262. In addition, the output device 260 may have a speaker or the like additionally. The input/output IF 270 is connected to the input device 250 and the output device 260.

The communication IF 280 connects the client apparatus 200 to the network 300 (or communication apparatus such as a router) communicatively. In other words, the client apparatus 200 is connected to the network 300 via a communication apparatus such as a modem, TA (Terminal Adapter) or a router, and a telephone line, or via a private line. In this way, the client apparatus 200 can access to the evaluating apparatus 100 by using a particular protocol.

The client apparatus 200 may be realized by installing softwares (including programs, data and others) for providing various kinds of processing functions included in the control device 210 to an information processing apparatus (for example, an information processing terminal such as a known personal computer, a workstation, a family computer, Internet TV (Television), PHS (Personal Handyphone System) terminal, a mobile phone terminal, a mobile unit communication terminal, or PDA (Personal Digital Assistants)) connected as needed with peripheral devices such as a printer, a monitor, and an image scanner.

All or a part of processings of the control device 210 in the client apparatus 200 may be performed by CPU and programs read and executed by the CPU. Computer programs for giving instructions to the CPU and executing various processings together with the OS (Operating System) are recorded in the ROM 220 or HD 230. The computer programs, which are executed as they are loaded in the RAM 240, constitute the control device 210 with the CPU. The computer programs may be stored in application program servers connected via any network to the client apparatus 200, and the client apparatus 200 may download all or a part of them as needed. All or any part of processings of the control device 210 may be realized by hardware such as wired-logic.

In the above-described present description of the configuration of the evaluating system, the evaluating apparatus 100 executes the reception of the concentration data, the evaluation for the individual based on the concentration data (including the conversion of the concentration value, the generation of the positional information, and the classification of the individual into the category), and the transmission of the evaluation results, while the client apparatus 200 executes the reception of the evaluation results, described as an example. However, when the client apparatus 200 includes the evaluating unit 210a, for example, the conversion of the concentration value, the generation of the positional information, and the classification of the individual into the category may be appropriately shared between the evaluating apparatus 100 and the client apparatus 200. For example, when the client apparatus 200 receives the converted value of the concentration value from the evaluating apparatus 100, the evaluating unit 210a may generate the positional information corresponding to the converted value in the generating unit 210a2 and classify the individual into any one of the categories using the converted value in the classifying unit 210a3. When the client apparatus 200 receives the converted value of the concentration value and the positional information from the evaluating apparatus 100, the evaluating unit 210a may classify the individual into any one of the categories using the converted value in the classifying unit 210a3.

### 2-3. Other Embodiments

In addition to the second embodiment described above, the evaluating apparatus, the evaluating method, the evaluating program product, the evaluating system, and the terminal apparatus according to the present invention can be practiced in various different embodiments within the technological scope of the claims.

Of the processings described in the second embodiment, all or a part of the processings described as automatically performed ones may be manually performed, or all or a part of the processings described as manually performed ones may be also automatically performed by known methods.

In addition, the processing procedures, the control procedures, the specific names, the information including parameters such as registered data of various processings and retrieval conditions, the screen examples, and the database configuration shown in the description and the drawings may be arbitrarily modified unless otherwise specified.

The components of the apparatuses constituting the evaluating system shown in the figures are functionally conceptual and therefore not be physically configured as shown in the figures.

For example, for the operational functions provided in the evaluating apparatus 100, in particular, for the operational functions performed in the control device 102, all or part thereof may be implemented by the CPU (Central Processing Unit) and programs interpreted and executed in the CPU, or may be implemented by wired-logic hardware. The program is recorded in a non-transitory tangible computer-readable recording medium including programmed instructions for making an information processing apparatus execute the evaluating method according to the present invention, and is mechanically read as needed by the evaluating apparatus 100. More specifically, computer programs to give instructions to the CPU in cooperation with the OS (operating system) to perform various processes are recorded in the memory device 106 such as ROM or a HDD (hard disk drive). The computer programs are executed by being loaded to RAM, and form the control unit in cooperation with the CPU.

The computer programs may be stored in an application program server connected to the evaluating apparatus 100 via an arbitrary network, and all or part thereof can be downloaded as necessary.

The evaluating program according to the present invention may be stored in the non-transitory tangible computer-readable recording medium, or can be configured as a program product. The “recording medium” mentioned here includes any “portable physical medium” such as a memory card, a USB (universal serial bus) memory, an SD (secure digital) card, a flexible disk, a magneto-optical disc, ROM, EPROM (erasable programmable read only memory), EEPROM (registered trademark) (electronically erasable and programmable read only memory), CD-ROM (compact disk read only memory), MO (magneto-optical disk), DVD (digital versatile disk), and Blu-ray (registered trademark) Disc.

The “program” mentioned here is a data processing method described in an arbitrary language or description method, and therefore any form such as a source code and a binary code is acceptable. The “program” is not necessarily limited to a program configured as a single unit, and, therefore, includes those dispersively configured as a plurality of modules and libraries and those in which the function of the program is achieved in cooperation with separate programs represented as OS (operating system). Any known configuration and procedures can be used as a specific configuration and reading procedure to read a recording medium by each apparatus shown in the embodiments, an installation procedure after the reading, and the like.

The various databases and the like stored in the memory device is a storage unit such as a memory device such as RAM and ROM, a fixed disk drive such as a hard disk, a flexible disk, or an optical disc. The memory device stores therein various programs, tables, databases, files for Web (World Wide Web) pages, and the like used to perform various processes and to provide Web sites.

The evaluating apparatus 100 may be configured as an information processing apparatus such as known personal computer and work station, or may be configured as the information processing apparatus connected to an arbitrary peripheral device. The evaluating apparatus 100 may be provided by installing software (including the programs and the data, etc.) to cause the information processing apparatus to implement the evaluating method according to the present invention.

Furthermore, a specific configuration of dispersion or integration of the apparatuses is not limited to the shown one. The apparatuses can be configured by functionally or physically dispersing or integrating all or part of the apparatuses in arbitrary units according to various types of additions or the like or according to functional loads. In other words, the embodiments may be implemented in arbitrary combinations thereof or an embodiment may be selectively implemented.

### Example 1

The blood samples of aged persons of 65 years old or older with normal cognitive function and the blood samples of aged persons diagnosed as AD were obtained (from a total of 70 people). The blood samples were measured by the measuring method (A) to determine the blood concentrations (mol/ml) of the 23 kinds of amino acids (α-ABA, Ala, Arg, Asn, Cit, Glu, Gln, Gly, His, Ile, Leu, Lys, Met, Orn, Phe, Pro, Ser, Thr, Trp, Tyr, Val, Cysteine, Taurine). Furthermore, the same blood samples were measured by the measuring method (A) to determine the blood concentration (mol/ml) of GABA.

For the blood concentrations of substances in the persons with normal cognitive function and the patients with AD, in the test with null hypothesis (Mann-Whitney U test) that “both groups have the same average value”, substances having a significant variation (p-value<0.05) were Taurine and GABA for the persons with normal cognitive function. In addition, significant variation was found in the values obtained by multiplying the blood concentrations of Taurine and GABA. Table 1 presents ROC AUC values of the receiver operating characteristic (ROC) curve used for discriminating persons with normal cognitive function and patients with AD based on the blood concentrations of the above-mentioned two substances and the values obtained by multiplying the blood concentrations. Example 1 proved that the above-mentioned two substances and the product of the blood concentrations thereof are valuable for evaluating the state of AD (for example, two-group discrimination for discriminating between AD patients and AD-free patients). ROC AUC is defined as the area under the curve (AUC) in the receiver operating characteristic curve (ROC) created by plotting (x, y)=(1-specificity, sensitivity) on two-dimensional coordinates. The value of ROC AUC is 1 in perfect discrimination, and the closer this value is to 1, the higher the discriminative characteristic.

TABLE 1

| SUBSTANCE NAME | ROC_AUC |
|---|---|
| Taurine | 0.8676 |
| GABA | 0.7712 |
| Taurine*GABA | 0.9069 |

### Example 2

The blood samples of aged persons of 65 years old or older with normal cognitive function and the blood samples of aged persons diagnosed as MCI were obtained (from a total of 60 people). The blood samples were measured by the same measuring method as Example 1 to determine the blood concentrations (mol/ml) of the 23 kinds of amino acids. Furthermore, the same blood samples were measured by the same measuring method as Example 1 to determine the blood concentrations (mol/ml) of the 11 kinds of amino acid related metabolites (GABA, Sarcosine, Serotonin, Spermidine, Phosphoethanoamine, S-Adenosyl-homocysteine, L-Homocitrulline, N8-Acetylspermidine, 3-hydroxykynurenine, Phosphoserine, Hypotaurine). The method proposed in 1995 by Petersen et al., from the Mayo clinic was used as the MCI diagnostic criteria (“Petersen, et al., Arch Neurol (1999) 56(6):760; Mild cognitive impairment: clinical characterization and outcome.”).

For the blood concentrations of substances in the persons with normal cognitive function and the patients with MCI, in the test with null hypothesis (Mann-Whitney U test) that “both groups have the same average value”, substances having a significant variation (p-value<0.05) were Met, Lys, Leu, Taurine, Sarcosine, GABA, Serotonin, Spermidine, Phosphoethanoamine, S-Adenosyl-homocysteine, L-Homocitrulline, N8-Acetylspermidine, 3-hydroxykynurenine, Phosphoserine, and Hypotaurine for the persons with normal cognitive function. Table 2 presents ROC AUC values of the ROC curve used for distinguishing between the persons with normal cognitive function and the patients with MCI based on the blood concentrations of the above-mentioned 15 substances. Example 2 proved that the above-mentioned 15 substances are valuable for evaluating the state of MCI (for example, two-group discrimination for discriminating between MCI patients and MCI-free patients).

TABLE 2

| SUBSTANCE NAME | ROC_AUC |
|---|---|
| Met | 0.6522 |
| Lys | 0.6967 |
| Leu | 0.6494 |
| Taurine | 0.7239 |
| Sarcosine | 0.8317 |
| GABA | 0.7556 |
| Serotonin | 0.9020 |
| Spermidine | 0.7911 |
| Phosphoethanolamine | 0.7456 |
| S-Adenosyl-homocysteine | 0.7317 |
| L-Homocitrulline | 0.6872 |
| N8-Acetylspermidine | 0.7378 |
| 3-hydroxykynurenine | 0.7204 |
| Phosphoserine | 0.6689 |
| Hypotaurine | 0.6650 |

### Example 3

The blood samples of aged persons of 65 years old or older with normal cognitive function and the blood samples of aged persons diagnosed as MCI were obtained (from a total of 60 people). The blood samples were measured by the same measuring method as Example 1 and Example 2 to determine the blood concentrations (mol/ml) of the 13 kinds of amino acid related metabolites (GABA, Sarcosine, Serotonin, Spermidine, Phosphoethanoamine, S-Adenosyl-homocysteine, L-Homocitrulline, N8-Acetylspermidine, 3-hydroxykynurenine, Phosphoserine, Hypotaurine, 3-Aminobutanoic acid, and Asymmetric dimethylarginine). The same method as Example 2 was used as the MCI diagnostic criteria.

For the blood concentrations of substances in the persons with normal cognitive function and the patients with MCI, in the test with null hypothesis (Mann-Whitney U test) that “both groups have the same average value”, substances having a significant variation (p-value<0.05) were 3-Aminobutanoic acid and Asymmetric dimethylarginine in addition to the substances listed in Table 2 for the persons with normal cognitive function. Table 3 presents ROC AUC values of the ROC curve used for distinguishing between the persons with normal cognitive function and the patients with MCI based on the blood concentrations of the above-mentioned two substances. Example 3 proved that the above-mentioned two substances are valuable for evaluating the state of MCI (for example, two-group discrimination for discriminating between MCI patients and MCI-free patients).

TABLE 3

| SUBSTANCE NAME | ROC_AUC |
|---|---|
| 3-Aminobutanoic acid | 0.8428 |
| Asymmetric dimethylarginine | 0.7844 |

### Example 4

The blood samples of aged persons of 65 years old or older with normal cognitive function and the blood samples of aged persons diagnosed as MCI were obtained (from a total of 30 people for each group). The blood samples were measured by the same measuring method as Example 1, Example 2, and Example 3 to determine the blood concentrations (mol/ml) and the peak area values of the 23 kinds of amino acids and 14 kinds of amino acid related metabolites (GABA, Sarcosine, Serotonin, Spermidine, Phosphoethanoamine, S-Adenosyl-homocysteine, L-Homocitrulline, N8-Acetylspermidine, 3-hydroxykynurenine, Phosphoserine, Hypotaurine, 3-Aminobutanoic acid, Asymmetric dimethylarginine, and Thioproline). The same method as Example 2 was used as the MCI diagnostic criteria.

For the blood concentrations of substances in the persons with normal cognitive function and the patients with MCI and the peak areas thereof, in the test with null hypothesis (Mann-Whitney U test) that “both groups have the same average value”, a substance having a significant variation (p-value<0.05) was Thioproline in addition to the substances listed in Tables 2 and 3 for the persons with normal cognitive function. Table 4 presents ROC AUC values of the ROC curve used for distinguishing between the persons with normal cognitive function and the patients with MCI using the peak area of Thioproline in blood. Example 4 proved that the concentration of Thioproline is valuable for evaluating the state of MCI (for example, two-group discrimination for discriminating between MCI patients and MCI-free patients).

TABLE 4

| SUBSTANCE NAME | ROC_AUC |
|---|---|
| Thioproline | 0.9905 |

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An evaluating method comprising:
an evaluating step of evaluating a state of mild cognitive impairment or Alzheimer's disease for a subject to be evaluated using a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, GABA, Hypotaurine, S-Adenosyl homocysteine, Sarcosine, Serotonin, Spermidine, hCit, 3-hKyn, aAiBA, N8-Acetylspermidine, Phosphoserine, bABA, ADMA, and Thioproline in blood of the subject.

2. An evaluating apparatus comprising a control unit, wherein the control unit includes:
an evaluating unit that evaluates a state of mild cognitive impairment or Alzheimer's disease for a subject to be evaluated using a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, GABA, Hypotaurine, S-Adenosyl homocysteine, Sarcosine, Serotonin, Spermidine, hCit, 3-hKyn, aAiBA, N8-Acetylspermidine, Phosphoserine, bABA, ADMA, and Thioproline in blood of the subject.

3. An evaluating method executed by an information processing apparatus including a control unit, wherein
the evaluating method comprises an evaluating step of evaluating a state of mild cognitive impairment or Alzheimer's disease for a subject to be evaluated using a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, GABA, Hypotaurine, S-Adenosyl homocysteine, Sarcosine, Serotonin, Spermidine, hCit, 3-hKyn, aAiBA, N8-Acetylspermidine, Phosphoserine, bABA, ADMA, and Thioproline in blood of the subject, wherein the evaluating step is executed by the control unit.

4. An evaluating program product having a non-transitory tangible computer readable medium including a programmed instruction for making an information processing apparatus including a control unit execute an evaluating method, wherein the evaluating method comprises an evaluating step of evaluating a state of mild cognitive impairment or Alzheimer's disease for a subject to be evaluated using a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, GABA, Hypotaurine, S-Adenosyl homocysteine, Sarcosine, Serotonin, Spermidine, hCit, 3-hKyn, aAiBA, N8-Acetylspermidine, Phosphoserine, bABA, ADMA, and Thioproline in blood of the subject.

5. An evaluating system comprising an evaluating apparatus including a control unit and a terminal apparatus including a control unit that are connected to each other communicatively via a network, wherein
the control unit of the terminal apparatus includes:
a concentration data-sending unit that transmits concentration data on a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, GABA, Hypotaurine, S-Adenosyl homocysteine, Sarcosine, Serotonin, Spermidine, hCit, 3-hKyn, aAiBA, N8-Acetylspermidine, Phosphoserine, bABA, ADMA, and Thioproline in blood of a subject to be evaluated, to the evaluating apparatus; and
a result-receiving unit that receives an evaluation result on a state of mild cognitive impairment or Alzheimer's disease for the subject, transmitted from the evaluating apparatus, and
the control unit of the evaluating apparatus includes:
a concentration data-receiving unit that receives the concentration data transmitted from the terminal apparatus;
an evaluating unit that evaluates the state of mild cognitive impairment or Alzheimer's disease for the subject using the concentration value of the at least one of the amino acids and the amino acid related metabolites included in the concentration data received by the concentration data-receiving unit; and
a result-sending unit that transmits the evaluation result obtained by the evaluating unit to the terminal apparatus.

6. A terminal apparatus comprising a control unit, wherein the control unit includes a result-obtaining unit that obtains an evaluation result on a state of mild cognitive impairment or Alzheimer's disease for a subject to be evaluated,
wherein the evaluation result is the result of evaluating the state of mild cognitive impairment or Alzheimer's disease for the subject using a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, GABA, Hypotaurine, S-Adenosyl homocysteine, Sarcosine, Serotonin, Spermidine, hCit, 3-hKyn, aAiBA, N8-Acetylspermidine, Phosphoserine, bABA, ADMA, and Thioproline in blood of the subject.

7. An evaluating apparatus comprising a control unit, being connected to a terminal apparatus communicatively via a network, wherein the control unit includes:
a concentration data-receiving unit that receives concentration data on a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, GABA, Hypotaurine, S-Adenosyl homocysteine, Sarcosine, Serotonin, Spermidine, hCit, 3-hKyn, aAiBA, N8-Acetylspermidine, Phosphoserine, bABA, ADMA, and Thioproline in blood of a subject to be evaluated, transmitted from the terminal apparatus;
an evaluating unit that evaluates a state of mild cognitive impairment or Alzheimer's disease for the subject using the concentration value of the at least one of the amino acids and the amino acid related metabolites included in the concentration data received by the concentration data-receiving unit; and
a result-sending unit that transmits an evaluation result obtained by the evaluating unit to the terminal apparatus.
